(19) United States
(12) Patent Application Publication
(10) Pub. No.: US 2014/0130810 A1
(43) Pub. Date: May 15, 2014

(54) SMART DRAPES FOR COLLISION AVOIDANCE

(71) Applicant: INTUITIVE SURGICAL OPERATIONS, INC., Sunnyvale, CA (US)

(72) Inventors: Mahdi Azizian, Sunnyvale, CA (US); Jonathan Sorger, Belmont, CA (US)

(21) Appl. No.: 14/079,227

(22) Filed: Nov. 13, 2013

Related U.S. Application Data
(60) Provisional application No. 61/726,430, filed on Nov. 14, 2012.

(51) Int. Cl. A61B 19/08

(52) U.S. Cl. CPC A61B 19/081
USPC 128/849

(57) ABSTRACT

Embodiments of a smart surgical drape are disclosed. The surgical drape includes an insulating material and one or more sensors mounted with the insulating material, the one or more sensors detecting proximity between the surgical drape and a device. Some embodiments of the smart surgical drape can be utilized on surgical robots or other devices in the surgical area to detect potential collisions.

### RELATED APPLICATIONS

This application claims priority to U.S. Provisional Application Serial No. 61/726,430, filed on Nov. 14, 2012, which is herein incorporated by reference in its entirety.

### TECHNICAL FIELD

Embodiments of the present invention are related to surgical drapes and, in particular, to smart drapes for collision avoidance.

### DISCUSSION OF RELATED ART

Surgical procedures can be performed through a surgical robot in a minimally invasive manner. The benefits of a minimally invasive surgery are well known and include less patient trauma, less blood loss, and faster recovery times when compared to traditional, open incision surgery. In addition, the use of robot surgical systems (e.g., teleoperated robotic systems that provide telepresence), such as the da ^{Vinci}® Surgical System commercialized by Intuitive Surgical, Inc. of Sunnyvale, Calif., is known. Such robotic surgical systems may allow a surgeon to operate with intuitive control and increased precision when compared to manual minimally invasive surgeries.

In a minimally invasive surgical system, a procedure is performed by a surgeon controlling the robot. The robot includes one or more instruments that are coupled to manipulator arms. The instruments access the surgical area through small incisions in the skin of the patient or through a natural orifice of the patient. In some situations, multiple robots may be utilized. In such instances, care needs to be taken to avoid collisions between those robots, which can be damaging to both the robots and any patients that may be undergoing a procedure.

Proposals for collision avoidance have included registration of the robots within the procedure room. This proposal requires a lengthy analysis of the room and takes a considerable amount of time. Further, such an analysis would require updates to ensure that errors do not occur and needs to be performed each time the room is reconfigured. Another proposed solution, specifically designed for the use of MRI imagers, involves optical fiber embedded into deformable covers on the MRI bore to detect collisions. However, this solution is complicated and expensive to implement.

Therefore, there is a need to develop better performing collision avoidance between robotic systems in a surgical environment.

### SUMMARY

In accordance with aspects of the present invention, a surgical drape includes an insulating material and one or more sensors mounted with the insulating material, the one or more sensors detecting proximity between the surgical drape and a device.

A method of providing collision avoidance according to some embodiments of the present invention includes providing at least one drape over at least a portion of a robot, the drape including one or more sensors; determining whether a collision with a device is probable based on the proximity or contact of the device with at least one drape; and sending a signal when it is determined that a collision is probable.

These and other embodiments are further discussed below with respect to the following figures.

### BRIEF DESCRIPTION OF THE DRAWINGS

FIG. 1 illustrates an example of a surgical environment that includes two robots.

FIG. 2 illustrates the use of smart drapes according to some embodiments of the present invention.

FIGS. 3A and 3B illustrate a smart drape according to some embodiments of the present invention.

FIGS. 4A, 4B, 4C, and 4D illustrate a smart drape with multiple proximity detectors according to some embodiments of the present invention.

FIG. 5 illustrates an operation of a capacitance based smart drape with multiple capacitive detectors according to some embodiments of the present invention.

FIGS. 6A and 6B illustrate inductive based proximity detectors according to some embodiments of the present invention.

FIG. 7 illustrates an embodiment of a sensor that utilizes a transmitter/detector type of proximity detector according to some embodiments of the present invention.

FIG. 8 illustrates an embodiment of a sensor that utilizes a pressure detector according to some embodiments of the present invention.

FIG. 9 illustrates an embodiment of a sensor that utilizes RFID technology according to some embodiments of the present invention.

FIG. 10 illustrates a smart drape that utilizes optical fiber according to some embodiments of the present invention.

### DETAILED DESCRIPTION

In the following description, specific details are set forth describing some embodiments of the present invention. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

This description and the accompanying drawings that illustrate inventive aspects and embodiments should not be taken as limiting—the claims define the protected invention. Various mechanical, compositional, structural, and operational changes may be made without departing from the spirit and scope of this description and the claims. In some instances, well-known structures and techniques have not been shown or described in detail in order not to obscure the invention.

Additionally, the drawings are not to scale. Relative sizes of components are for illustrative purposes only and do not reflect the actual sizes that may occur in any actual embodiment of the invention. Like numbers in two or more figures represent the same or similar elements.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figure is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special device positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

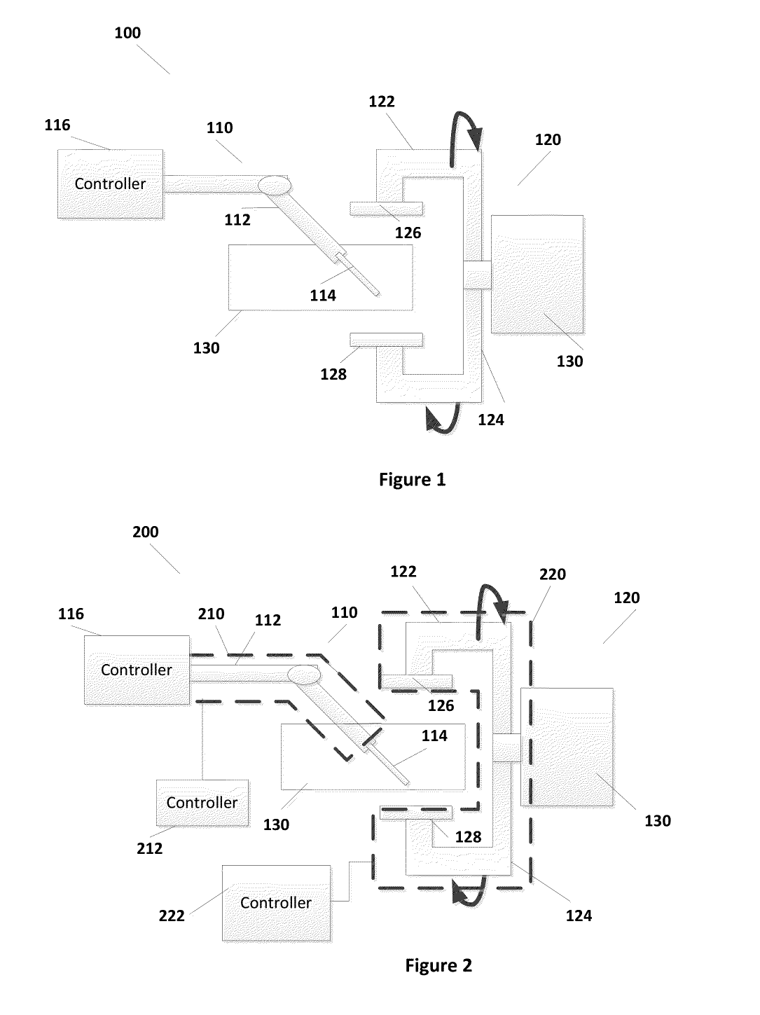

FIG. 1 illustrates a surgical environment 100. Surgical environment 100 includes a surgical robot 110 and an imager 120. As shown in FIG. 1, surgical robot 110 includes an articulating arm 112 attached to a surgical instrument 114. Surgical instrument 114 can be a single manipulator instrument, for example in a multi-port robotic system, or include multiple manipulator instruments, for example for a single port robotic system. Surgical robot 110 can be controlled by a controller 116. Controller 116 can manipulate articulating arm 112 and surgical instrument 114, either under autonomous control or according to input from a surgeon. Alternatively, articulating arm 112 may be moved manually during a procedure or procedure set-up.

In addition to surgical robot 110, surgical environment 100 can include imager 120. Imager 120 can be, for example, an x-ray computed topography imager (a CT imager), or other imaging technology. In some embodiments, imager 120 can include a second surgical robot. In general, imager 120 can include a controller 130, support arms 122 and 124, source 126, and detector 128. Source 126 and detector 128 can be attached to support arms 122 and 124, respectively, as shown or other arrangements may be used. Imager 120 can rotate arms 122 and 124 around surgical table 130 such that imager 120 can provide enough data to controller 130 to compile an image of the surgical area. In some embodiments, the rotational speed of arms 122 and 144 can be rather large (e.g. imaging robot 120 may, for example, make one revolution every 3 seconds or faster).

Collision of arms 122 and 124 with arm 112 of surgical robot 110 can be damaging to both surgical robot 110 and imaging robot 120. Additionally, in the likely event that surgical instrument 114 is inserted into a patient (not shown), then injury to the patient also likely results.

FIG. 2 illustrates a surgical environment 200 according to some embodiments of the present invention. Surgical environment 200 includes surgical robot 110 and imaging robot 120, as did surgical environment 100. However, in surgical environment 200, a drape 210 covers an operative portion of surgical robot 110 and a drape 220 covers an operative portion of imager 120. Drape 210 and drape 220 can be sterile drapes. Some examples of sterile drapes that can be utilized are discussed, for example, in U.S. Pat. No. 8,202,278, issued on Jun. 19, 2012, and U.S. Pat. No. 8,206,406, issued on Jun. 26, 2012, both of which are herein incorporated by reference in their entirety. Other sterile drapes can also be utilized. In general, drapes 210 and 220 can be blanket-like devices that are positioned to cover articulating arm 112 of surgical robot 110 and rotating arms 122 and 124 of imaging robot 120, respectively. Although both drapes 210 and 220 are illustrated in FIG. 2, some embodiments of surgical environment 200 may include one of drapes 210 and 220 and not both of them.

In general, drapes according to the present invention can be utilized with any portion of the area in which the robots are being deployed. Drapes can be utilized to cover instruments, patients and other personnel, or any other portion of the area.

As shown in FIG. 2, one or both of surgical drapes 210 and 220 are smart drapes. As such, in the example of FIG. 2, surgical drape 210 is coupled to controller 212 and surgical drape 220 is coupled to controller 222. Surgical drape 210 and surgical drape 220, either separately or operating together, include proximity or contact sensing. As such, controllers 212 and 222 can sense the proximity or contact between surgical robot 110 and imaging robot 120 and, in the event of an imminent collision or an actual collision, can communicate that collision event to one or both of controllers 116 and 130. A collision event, for example, can be sensed when one of surgical drapes 210 and 220 senses an object or the other of drapes 210 and 220 to be within a threshold distance. The threshold distance can be predetermined, may be physical contact, or may depend on known predicted motions of the draped robots. In the event of an imminent or actual collision as determined by the sensing of a collision event, motion of robot 110 and robot 120 can be halted. As such, an actual collision can be prevented or, in the event of actual contact, damage can be avoided or reduced.

As is discussed further below, one or both of drapes 210 and 220 provide for proximity sensing or contact sensing. Such sensing can include capacitive, conductive, inductive, acoustic, pressure, optical, radio frequency identification (RFID), shape, or some other sensing mechanism that allows for the determination of distance or actual contact. Drapes 210 and 220 can communicate with independent controllers 212 and 222, or with a single controller that combines both controllers 212 and 222. In some sensing technologies, two smart drapes are utilized and in some technologies only a single smart drape is utilized. In some environments, drapes can be placed on other components, including, but not limited to the surgical table and patient.

Once contact is measured or a potential collision is detected, then controllers 116 and 130 can be triggered to halt motion. In some embodiments, when a smart drape, for example drape 210, measures a distance to another object that is within a specified threshold difference, robots 110 and 120 are halted. In some cases, the specified distance may be actual contact. Several examples of proximity or contact sensing drapes are discussed below.

Drapes 210 and 220 can be applied to robots 110 and 120 similarly to other surgical drapes. Drapes 210 and 220 may include straps or other devices to attach them to robots 110 and 120. Any attachment device, for example utilization of snaps mounted on the robots, Velcro®, buckles, or other devices may be utilized to secure drapes 210 and 220 onto robots 110 and 120, respectively.

Electrical connections between drape 210 and controller 212 or between drape 220 and controller 222 can be accomplished in many ways, including through the use of standard electrical connectors, wireless communications, and digital communications methods. Drapes 210 and 220 can be sterilized, for example with conventional methods, and may be disposable. Drapes 210 and 220, in addition to providing the function of collision detection, may still provide the function of providing a sterile environment for the surgical area. In that fashion, in some embodiments surgical instruments associated with manipulators 114 are loadable during a surgical procedure. In some embodiments, drapes 210 and 220 can be smaller cuffs that fit around articulating arm 112 or on imaging robot 120 and positioned at the most likely collision location. In some applications, conventional drapes can be utilized in combination with the smart drapes.

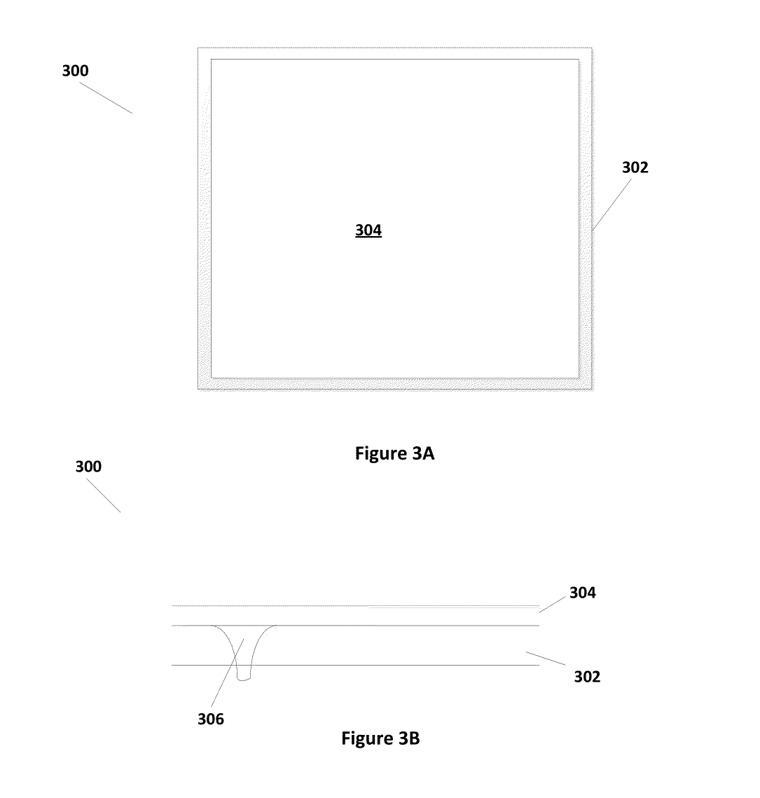

FIGS. 3A and 3B illustrate a smart drape 300 according to some embodiments of the present invention. As shown in FIGS. 3A and 3B, smart drape 300 includes a conductive material 304 fixed onto an insulating material 302. Insulating material 302 can be formed of a material configured to effectively shield a robot (for example surgical robot 110 or imaging robot 120) from the surgical site so that most of the components of the surgical robot do not have to be sterilized prior to, or following, the surgical procedure. Insulating material 302 may be multi-layered and may be similar to conventional sterile drapes.

As shown in FIG. 3A, conductive material 304 can be attached to insulating material 302 such that drape 300 can be applied to an instrument such as surgical robot 110 or imaging robot 120. As indicated, conductive material 304 may be flexible so that drape 300 can be formed over the instrument as needed.

In operation, conducting layer 304 can be utilized as a proximity sensor. For example, conducting layer 304 can be charged and its voltage monitored. When conducting layer 304 contacts another grounded conductor, then that grounding can be sensed by the voltage on conductor 304. For example, in FIG. 2 if drape 210 is drape 300 as shown in FIG. 3, then contact with imaging robot 120, where arms 122 and 124 are grounded, will be sensed by controller 212 and that information utilized in either controller 116 or controller 130 to stop the motion. If a drape 220 is utilized that is also constructed as drape 300, then conducting layer 304 of drape 220 can be grounded.

In another operation, if both drape 210 and drape 220 are constructed as drape 300, then the capacitance between the conducting layer 304 of drape 210 and the conducting layer 304 of drape 220 can be monitored. In some embodiments, a voltage (either direct-current or alternating current) can be applied between drapes 210 and 220. The capacitance will vary as the distance between drapes 210 and 220. Therefore, a potential collision can be sensed by controllers 212 and 222 prior to actual contact between surgical robot 110 and imaging robot 120.

As is further shown in FIG. 3B, a metallic clip 306 can be formed through insulator 302. Clip 306 can mate with a similar device positioned on the instrument to provide electrical contact. Clip 306 can be part of a snap fastener that can help keep drape 300 in place. The female portion of the snap fastener may be insulating from the remainder of the instrument and may include wiring to a controller as shown in FIG. 2. In some embodiments, the female portion of the snap fastener may be grounded so that conductor 304 is grounded. Other connectors can be utilized as well.

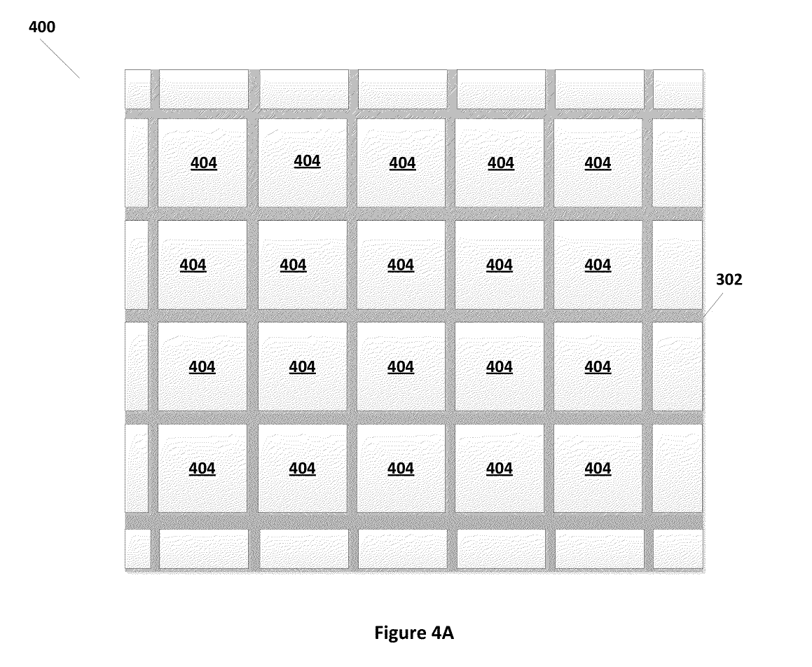

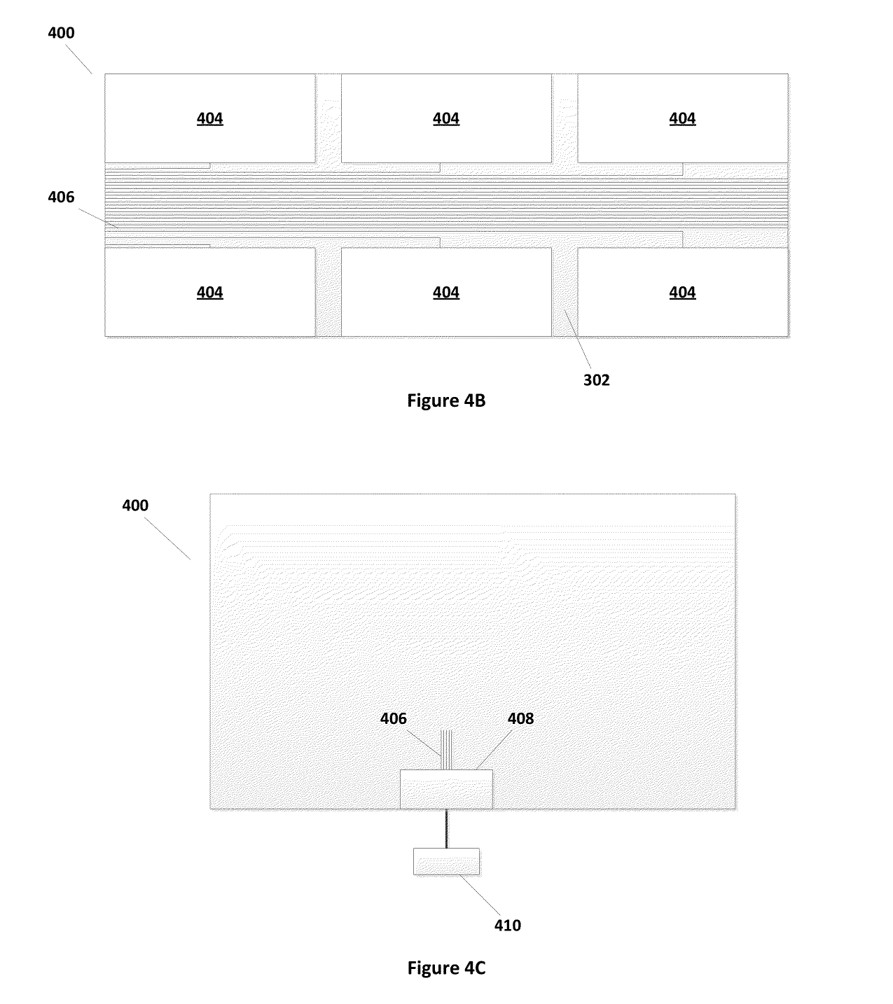

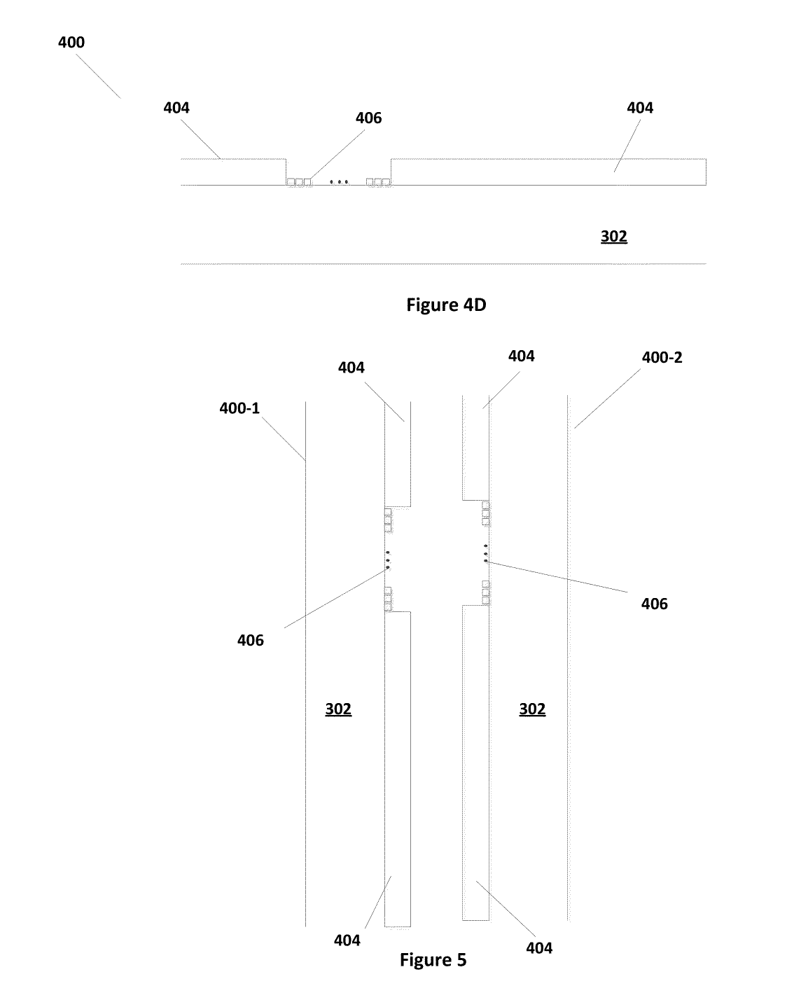

FIGS. 4A, 4B, 4C, and 4D illustrate a drape 400 that can be utilized as drape 210 or drape 220 as shown in FIG. 2. As illustrated in FIG. 4A, drape 400 includes sensors 404, which are arranged in an array of sensors 404 on insulator 302. Sensors 404, although illustrated as squares in FIG. 4A, can be of any shape and size. Additionally, although illustrated as arranged in a two-dimensional array, sensors 404 can be strips in a one-dimensional array. Further, sensors 404 can be of any type of proximity sensors. Having an array of sensors 404 as illustrated in FIG. 4A allows a more accurate determination of where on drape 400 a collision may occur, which correlates to where on an instrument the collision may occur.

In some embodiments one or more clips 306 can be utilized with each of sensors 404 to provide for electrical contact through insulating layer 302 to sensors 404. FIG. 4B illustrates another embodiment where wiring 406 is arranged between sensors 404. As shown in FIG. 4B, wiring 406 can be provided between rows or columns of sensors 404. Wiring 406 provides electrical connections to each of sensors 404. Wiring 406 can provide power and driving signals to sensors 404 as well as receiving signals from sensors 406. Although drape 400 illustrated in FIG. 4A shows an array of sensors 404, sensors 404 can include both transmitters and receivers. For example, sensors 404 can include both optical transmitters and optical receivers for optical sensing or acoustic transmitters and acoustic receivers for acoustic (e.g. ultrasonic) sensing. Furthermore, each of sensors 404 may include an optical indicator (e.g., may be coated with an OLED or other such device) to indicate visually where a contact has been made or a collision is about to occur.

FIG. 4C illustrates a controller 408. Controller 408 can be electrically coupled to each of sensors 404 through wiring 406. In some embodiments, controller 408 can process signals from sensors 404, for example by providing analog-to-digital conversion and serialization into a single data stream, and transmit the signals through connector 410. Connector 410 can be any of the standard electrical or optical connectors. In some embodiments, controller 408 can transmit signals wirelessly. Controller 408, therefore, transmits signals from sensors 404 to a drape controller. If controller 408 is, for example, drape 110, then the drape controller is controller 212. The drape controller (e.g., controller 212 or controller 222 shown in FIG. 2) can then process the signals to determine whether there is a collision.

FIG. 4D illustrates a cross section of some embodiments of drape 400. As shown in FIG. 4D, wiring 406 is positioned in the spacing between two of sensors 404. Wiring 406 can be included as individual shielded wires or can be conducting strips attached to insulator 302 that are connected to individual ones of sensors 404 and to controller 408 shown in FIG. 4C.

In some embodiments of drape 400, individual ones of sensors 404 can be selectively activated. Referring to FIG. 2, controller 212 in communications with controller 116 or controller 130 may utilize the kinematic information from surgical robot 110 or imaging robot 120, respectively, to predict areas where there is a higher likelihood of a collision and activate individual sensors 404 that correspond to those areas. Other ones of sensors 404 may be inactive. In some embodiments, instead of deactivating sensors in an area with a low likelihood of collision, sensors in the areas with a higher likelihood of collision may be sampled more frequently than sensors in an area with a lower likelihood of collision. Such arrangements may result in less data processing and consequently a faster response time to a contact or potential collision condition.

FIG. 5 illustrates an embodiment where two drapes 400 are in close proximity with one another and where sensors 404 are conductors. In that case, then each sensor 404 on drape 400-1 and one or more sensors 404 on drape 400-2 interact. The capacitance measured between each sensor 404 on drape 400-1 and sensors 404 on drape 400-2 provide an indication of the distance between drapes 400-1 and 400-2. Consequently, a controller coupled to monitor the capacitance between sensors 404 of drape 400-1 and sensors 404 of drape 400-2 can determine whether or not a collision is imminent between drapes 400-1 and 400-2.

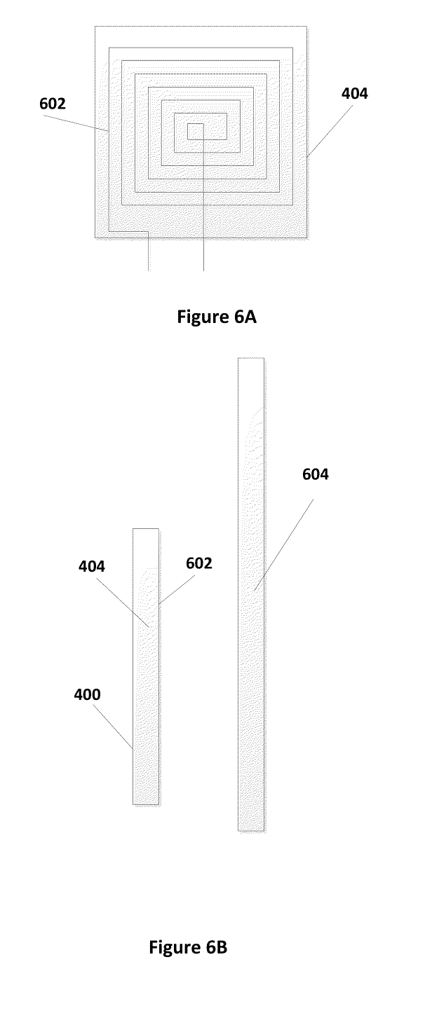

FIG. 6A illustrates an embodiment of sensor 404. The embodiment of sensor 404 illustrated in FIG. 6A includes a coil 602. Coil 602 can be utilized, for example, in an eddy current proximity sensor. In an eddy current proximity sensor, coil 602 is driven with an AC signal. The AC signal induces currents in a metallic surface that is placed in proximity to sensor 404. The magnetic field produced by the induced current can be measured at coil 602, leading to an indication of the distance between sensor 404 and the metallic surface. FIG. 6B illustrates this concept. Sensor 404 with a coil 602 is placed opposite a material 604. In this example, material 604 is a conductor. Material 604, for example, can represent a surgical robot with a metallic housing or it can represent a drape such as that illustrated in FIG. 3A.

In another example, coil 602 can be utilized to inductively measure a magnetic field produced by an opposing coil that is driven by an AC signal. In this example, material 604 includes a drape with sensors 404 that include coils 602 as illustrated in FIGS. 4A and 6A. Coils 602 of material 604 are driven in a known fashion. The electromagnetic fields produced by coils 602 of material 604 are then detected by coils 602 of sensors 404 in drape 400. Consequently, the distance between drape 400 and material 604 can be determined by the strength of the measured field. As discussed above, because drape 400 is tiled, a location of closest approach of material 604 to drape 400 can also be determined.

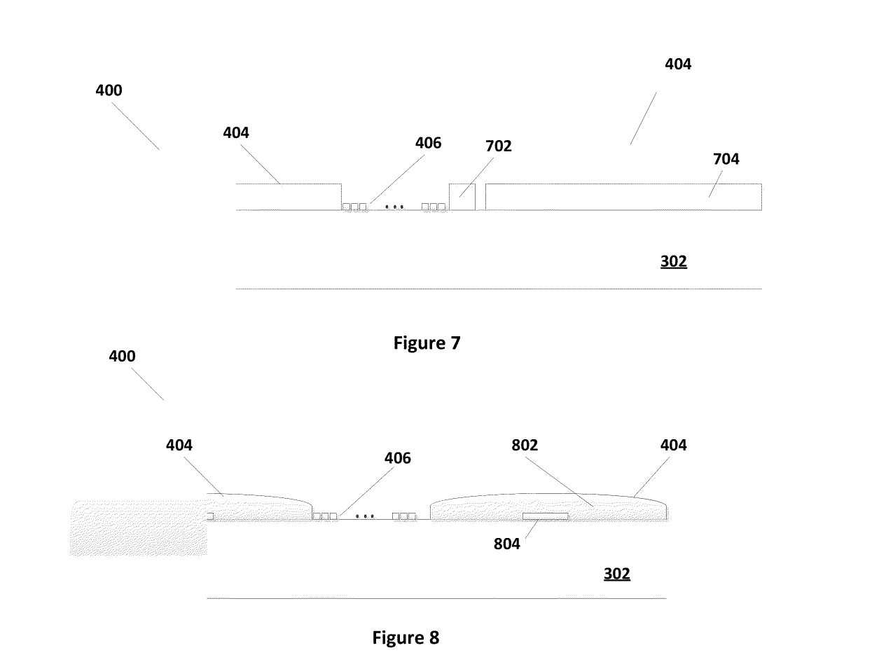

FIG. 7 illustrates a sensor 404 that includes both a transmitter 702 and a detector 704. The example of sensor 404 shown in FIG. 7 can, for instance, be acoustic or optical in nature. For example, transmitter 702 can be an LED while detector 704 can detect the reflected light emitted by LED detector 704. In that case, a distance between sensor 404 and a reflective surface can be determined. Similarly, transmitter 702 can be an acoustic transducer such as a piezoelectric material and detector 704 can be an acoustic sensor. In some embodiments, transmitter 702 and detector 704 can be combined so that, for example, a single piezoelectric acoustic detector can be utilized for transmission and detection. In either case, the distance to an object that reflects the acoustic signal can be determined by transmitting an acoustic signal and monitoring its reflected signal. As shown in FIG. 7, wiring 406 can include driving wires that supply driving voltages to transmitter 702 as well as signal lines that receive signals from detector 704.

FIG. 8 illustrates a sensor 404 that is a pressure sensor. Sensor 404 includes a cushion 802 with a pressure sensor 804. Pressure sensor 804 can, for example, be a piezoelectric material, which provides an electrical signal related to the pressure in cushion 802. Cushion 802 can, for example, be an air pocket or filled with a gel. In addition to detecting actual contact between drape 400 and an object, cushion 802 can help to deflect the severity of such a collision.

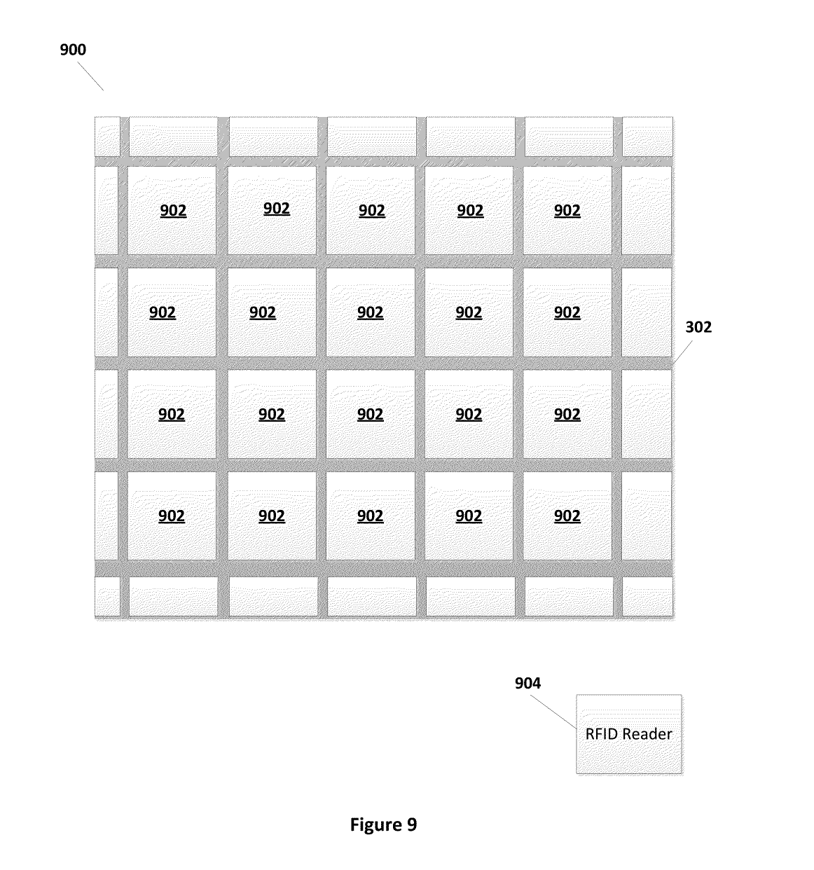

FIG. 9 illustrates a drape 900 that includes an array of RFID devices 902. RFID devices 902 can be mounted on or embedded into insulating layer 302. Again, RFID devices 902 can communicate with an RFID reader on an instrument to determine the location and orientation of drape 900 relative to RFID reader 904. RFID reader 904 can be RFID devices 902 on another drape 900 or can be a reader mounted on another robotic instrument or elsewhere in the operating room.

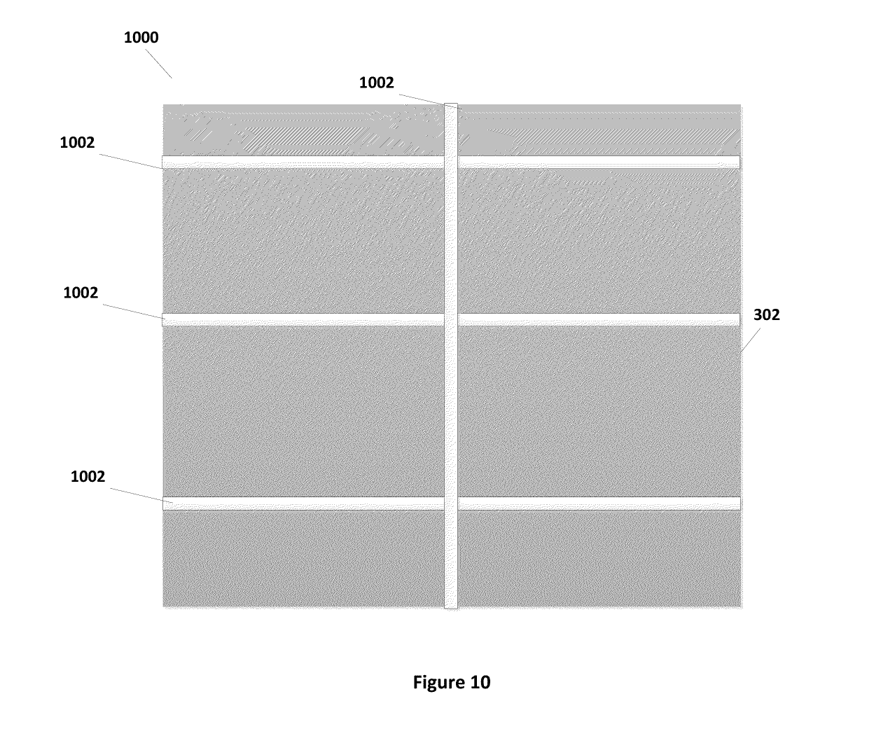

FIG. 10 illustrates a drape 1000 that includes shape sensing optical fiber 1002. Shape sensing optical fiber 1002 can be obtained, for example, from Luna Innovations Incorporated, 1 Riverside Circle, Suite 400, Roanoke, Va., 24016. Shape sensing optical fiber 1002 can be utilized to determine with a high level of accuracy the shape of optical fiber 1002 along its entire length. As a result, any distortion of drape 1000 from a baseline shape can be detected by optical fibers 1002. There can be any number of optical fibers 1002 and they may be oriented in any fashion to best determine when drape 1000 has been disturbed. The results can indicate when an object has come into contact with drape 1000 and thereby indicated a collision.

The above detailed description is provided to illustrate specific embodiments of the present invention and is not intended to be limiting. Numerous variations and modifications within the scope of the present invention are possible. The present invention is set forth in the following claims.

What is claimed is:

1. A surgical drape, comprising:
a drape material; and
one or more proximity sensors mounted on the insulating material, the one or more proximity sensors configured to detect proximity between the drape material and a device.

2. The surgical drape of claim 1, wherein the one or more proximity sensors includes a single conducting layer.

3. The surgical drape of claim 1, wherein the one or more proximity sensors includes an array of conducting layers.

4. The surgical drape of claim 2, wherein electrical connection to the single conducting layer is provided through one or more clips in the drape material.

5. The surgical drape of claim 2, wherein electrical connection to the array of conducting layers is provided through one or more clips in the drape material.

6. The surgical drape of claim 1, wherein the one or more proximity sensors includes at least one conducting layer and a capacitance is measured between the at least one conducting layer and the device.

7. The surgical drape of claim 1, wherein the one or more proximity sensors each includes a conducting layer and a capacitance is measured between each of the conducting layers and the device.

8. The surgical drape of claim 1, wherein the one or more proximity sensors include coils.

9. The surgical drape of claim 8, wherein the one or more proximity sensors are driven, and measurement of proximity to the device is performed utilizing induced currents.

10. The surgical drape of claim 8, wherein the one or more proximity sensors detect an electromagnetic field generated at the device.

11. The surgical drape of claim 1, wherein the one or more proximity sensors each include a transmitter and a receiver.

12. The surgical drape of claim 11, wherein the transmitters are acoustic and the receivers detect acoustical energy reflected from the device.

13. The surgical drape of claim 11, wherein the transmitters are optical and the receivers detect optical energy reflected from the device.

14. The surgical drape of claim 1, wherein the one or more proximity sensors include a cushion with a pressure sensor configured to sense pressure in the cushion, the one or more proximity sensors detecting contact with the device.

15. The surgical drape of claim 1, wherein the one or more proximity sensors include radio frequency identification devices.

16. The surgical drape of claim 1, wherein the one or more proximity sensors include shape sensing optical fiber.

17. The surgical drape of claim 1, further including a sampling unit that samples at least one proximity sensor of the one or more proximity sensors at a low frequency based on a determination of a probable location for a collision.

18. A method of operating a robot, comprising:
moving the robot, wherein at least a portion of the robot is covered by a drape including one or more proximity sensors;
determining a proximity of a device with the at least one drape using the one or more proximity sensors; and
sending a signal when the proximity reaches a threshold value.

19. The method of claim 18, wherein the one or more proximity sensors include conductors.

20. The method of claim 17, wherein the one or more proximity sensors include capacitive proximity sensing.

21. The method of claim 18, wherein the one or more proximity sensors include inductive proximity sensors.

22. The method of claim 18, wherein the one or more proximity sensors include acoustic proximity sensors.

23. The method of claim 18, wherein the one or more proximity sensors include optical proximity sensors.

24. The method of claim 18, wherein the one or more proximity sensors include shape sensitive optical fiber.

25. The method of claim 18, including
predicting which of the one or more sensors are likely to be in an area of a collision; and
sampling sensors that are less likely to be in the area of a collision at a lower frequency than sensors that are in the likely area of collision or deactivating sensors with less likelihood of collision.
